(19) United States
(12) Patent Application Publication
(10) Pub. No.: US 2025/0040936 A1
(43) Pub. Date: Feb. 6, 2025

(54) COMPRESSION FEEDBACK HEMOSTASIS DEVICE

(71) Applicant: Biolife, L.L.C., Sarasota, FL (US)

(72) Inventors: Talmadge Kelly Keene, Ruskin, FL (US); Kurt Vadelund, Apollo Beach, FL (US); Kenneth Wiglund, Bradenton, FL (US); John Tifton Fordham, Indialantic, FL (US)

(21) Appl. No.: 18/787,249

(22) Filed: Jul. 29, 2024

Related U.S. Application Data
(60) Provisional application No. 63/626,375, filed on Jan. 29, 2024.
(60) Provisional application No. 63/530,151, filed on Aug. 1, 2023.

(51) Int. Cl. A61B 17/132; A61B 17/12; A61F 13/00; A61F 13/02

(52) U.S. Cl. CPC A61B 17/1322; A61F 13/00059; A61F 13/0273; A61B 2017/12004; A61F 2013/0028

(57) ABSTRACT

An audible and/or tactile feedback hemostasis compression device employs a mechanism that produces a sound or tactile response when sufficient force is applied atop a wound post vascular procedure and applies proper hemostasis compression. This device can be used with circumferential bands, manual pressure, or other mechanical compression devices to create more uniform band securement, resulting in more uniform wound compression.

### RELATED APPLICATION

The present application claims the benefit of U.S. Provisional Patent Application No. 63/530,151, filed on Aug. 1, 2023, and U.S. Provisional Patent Application No. 63/626,375, filed on Jan. 29, 2024, the entire disclosure of which is incorporated by reference herein.

### FIELD OF THE INVENTION

The present invention relates in general to hemostasis, methods of achieving hemostasis, devices used in such methods, and in particular, to hemostasis of a blood vessel immediately after performing a vascular catheterization procedure.

### BACKGROUND OF THE INVENTION

Many medical procedures that once required extensive invasive surgery are performed today less invasively by inserting surgical or diagnostic devices through arteries or veins (i.e., vascular procedures). These procedures are much safer and require significantly less recovery time than prior procedures. To prevent clots from forming in the vessels during and after the procedure, the patient may require anticoagulation medications, which often result in excessive bleeding. To stop bleeding after vascular procedures, direct pressure is typically applied to the wound. This pressure must be held over both the entry point wound in the skin and the wound that was created in the vessel. For example, after a catheterization lab procedure (e.g., angiograms) there is a puncture wound created in the skin and in the artery that can both bleed. Unlike a femoral procedure, because the radial artery is near the surface of the skin the punctures are normally close together and surface compression is more easily translated to compression over the arteriotomy.

Such direct pressure can be applied manually by a clinician, but in many instances the pressure is needed for an extended period to decrease or stop the bleeding. Early hemostasis over the radial artery was accomplished using regular gauze to create a compression bandage. To save the clinicians time, prevent clinician injuries (e.g., carpel tunnel) and to improve on consistency, many compression devices have been developed. Some of such devices employ a pressure dressing or an inflatable bladder or balloon with a circumferential band to create the compression needed to stop the bleeding. First the band is wrapped around the limb or other body part, and then the bladder is inflated to create compression pressure over the wound site. As stated previously, the compression must cover both the wound in the skin as well as the wound in the vessel beneath the skin. Bands typically employ a more rigid structure on the distal side of the balloon that allows the balloon to push against to apply pressure to the patient.

The most popular currently marketed band (TR Band®) consists of a syringe for inflating; a check valve to hold air in the balloon once inflated; a tube connecting the check valve to the balloon; a circumferential band employing Velcro® as an attachment method to hold the band in place around the limb of the patient; and an inflatable balloon. However, no devices currently on the market provide a feedback mechanism to the clinician to indicate the amount of compression being applied over the artery at a given compression level.

There are multiple ways to create the compression over the audible/tactile feedback device. Some of these concepts are described below. This is not an all-encompassing list and is only meant to explain the current technology. The bands can be secured around the limb with hook and loop attachments (e.g., Velcro), a buckle, ziptie technology, adhesive dressing, cohesive dressings, or other methods. Compression can be created by the level of securement of the band over compression dressing, a balloon to create depression, or a screw mechanism. How the band is secured around the limb is not important; what is important is creating uniform securement tightness employing the audible/tactile feedback hemostasis compression device to set the proper compression needed over a bleeding wound.

Recent testing has revealed that the variabilities in band securement or tightness around the limb has an impact on the compression applied as the balloon is inflated. A tighter band prior to inflation, on the same patient with the same volume of air, will create more compression than will a more loosely applied band.

Currently, bands are placed with significant variation in tightness when secured around a limb which creates massive variation in compression force over an artery with volumetric inflation of the balloon. Too little pressure will result in bleeding and/or hematomas and too much pressure for too long may result in radial artery occlusion in the healthcare setting. These lead to extended patient stays, overnight stays, and increases in healthcare costs.

Further, multiple variables can make training clinicians for band attachment a difficult and inconsistent process. These factors make it difficult to create and apply a standard volumetric inflation hemostasis protocol. An unexpected solution to this issue has been found using a band attached to a wrapping dressing over a rigid or semi-rigid device to apply pressure over the wound that employs a mechanism that provides audible/tactile feedback as the device is compressed to the proper level.

### SUMMARY OF THE INVENTION

This invention relates generally to employing a device for use with a circumferential wound cover to apply pressure over a wound in order to create hemostasis. In addition, there is a mechanism that provides audible/tactile feedback when the desired compression force is achieved. The feedback can be mechanical or electronic in nature.

The present invention addresses a method of creating consistent pressure on both wounds from patient to patient that is not possible with any current compression band system on the market, by means of adding an audible/tactile feedback mechanism for the clinician.

The novelty of this new device will create significantly more uniform securement of the bands around a patient's limbs, resulting in more consistent compression over the wound and a better method of obtaining hemostasis.

For patients with elevated blood pressure additional compression past the level indicated by the feedback mechanism may be necessary, still the feedback mechanism will help the clinician create the higher level of compression by giving them a relationship between the band “tightness” and that amount of compression created over the bleeding site.

In one particular embodiment, the invention is a device for use with a circumferential wrap, band, or other methods of applying compression over a bleeding wound wherein the device is rigid or semi-rigid and has a patient contact area of 0.5 to 10 square inches comprising a top portion with a wrap contact surface or attachment mechanism to allow the creation of audible and/or tactile feedback at an applied compression force of 50 to 300 mmHg at the patient contact area.

Another embodiment is a band in conjunction with a balloon to create compression force over the bleeding wound. In an embodiment, these types of bands are the primary device used for radial compression after a radial artery vascular access procedure.

### BRIEF DESCRIPTION OF THE DRAWINGS

FIG. 1 is a photograph of a children's toy clicker device purchased for the purpose of testing in the context of the present invention.

FIG. 2 is a photograph of a prototype device created using the device of FIG. 1 and a 3D printed housing.

FIG. 3 are photographs of self-adhering elastic wrap being applied over the prototype device of FIG. 2 to create compression over the radial artery.

FIG. 4 is a photograph of a Velcro band being applied over the prototype device of FIG. 2 to create compression over the radial artery.

FIG. 5 is an image of a mechanical device, namely the CompressAR® device, which may be used in conjunction with the present invention.

FIG. 6 shows the results of compression measure testing using the silicone arm model of FIG. 2 with a load cell inserted at the radial artery position.

FIG. 7 is a table showing the results of compression created from the compression measure testing results of FIG. 5.

FIG. 8 is a graph comparing the compression created by the protype device of FIG. 2 (black graph) with three market leading bands using a 2-step deflation protocol.

FIG. 9 is a prototype balloon band device incorporated into a self-adhering elastic wrap.

FIG. 10 is the prototype device of FIG. 8 wrapped around a wrist of a patient.

FIG. 11 is a graph demonstrating a band filled with air and tested over time for air loss.

FIG. 12 is a graph demonstrating procedure to stabilize any relaxation irregularities that the device may show over time.

### DETAILED DESCRIPTION OF PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. The following embodiments and aspects thereof are described and illustrated in conjunction with systems, tools and methods which are meant to be exemplary and illustrative and not limiting in scope. In various embodiments one or more of the above-described problems have been reduced or eliminated while other embodiments are directed to other improvements.

Thus, it is intended that the present invention covers such modifications and variations that come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

The present invention is directed, in an embodiment, to a device for creating improved hemostasis, wherein the device comprises a circumferential band or wrap, a device to apply pressure over the wound, and a mechanism to create audible and/or tactile feedback. The audible/tactile feedback device would be placed around the patient's limb, secured until the mechanism provides audible/tactile feedback, left secured to the patient's wound area at the desired level of compression, and then monitored for adherence to the desired level of compression.

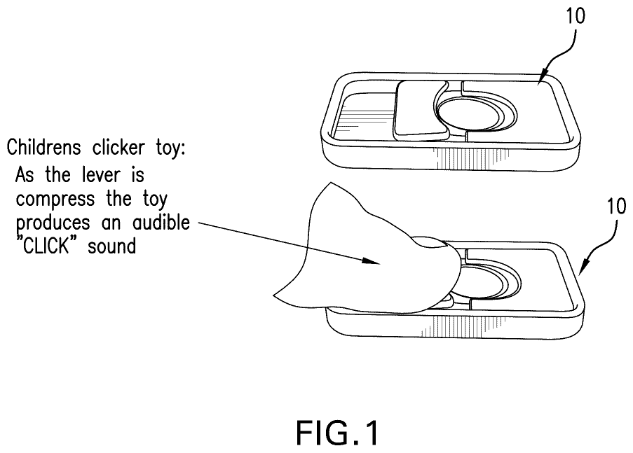

Generally, there are many children's toys and dog training devices on the market that utilize a mechanism that creates audible and/or tactile feedback as the device is compressed. FIG. 1 is an example of a toy clicker device 10 that was purchased for producing a prototype of the present invention. As shown in FIG. 1, as the lever of the toy is compressed, the toy produces an audible “click” sound.

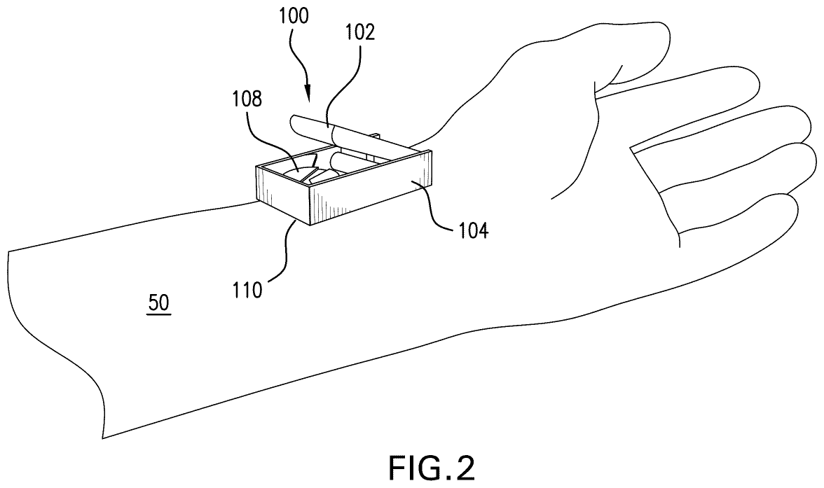

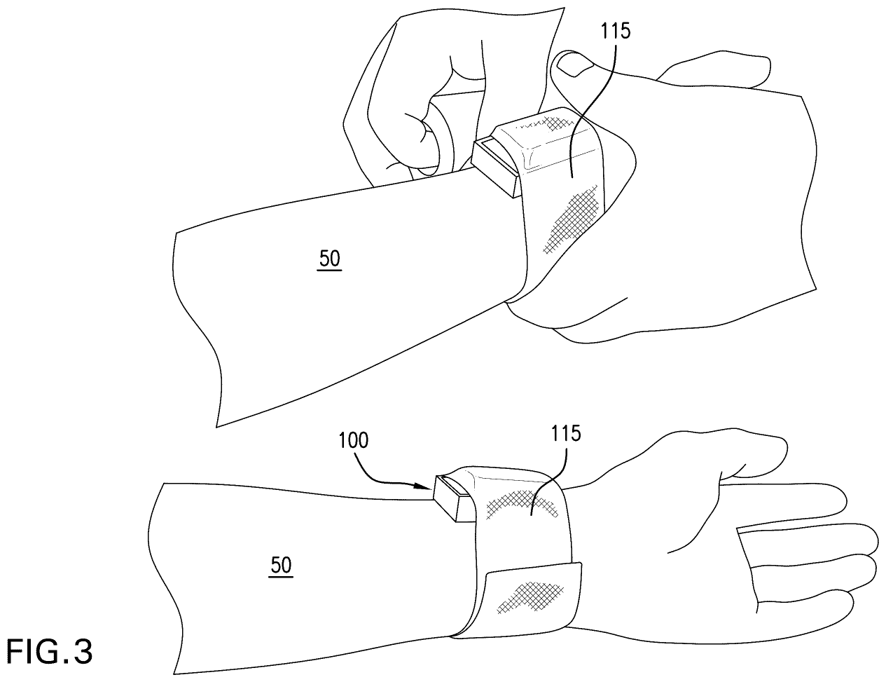

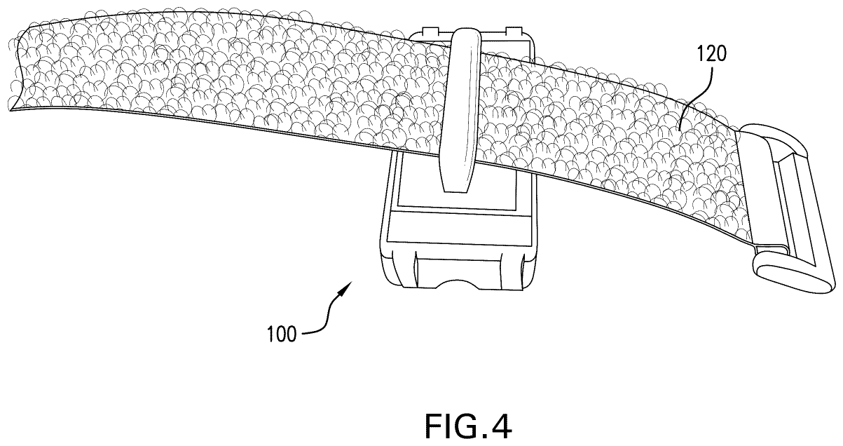

In testing, a prototype device 100 was created from a clicker toy mechanism and a 3D printed housing 104 as shown in FIGS. 2-4. The clicker portion 108 of the toy shown in FIG. 1 was removed and inserted into the patient contact portion 110 of the device 100, i.e., the portion of the device 100 that has contact with the patient, as shown in FIG. 2. A rocker arm wrap contact top 102 was then added to the top of the depressible lever. The created prototype was then secured and applied to a silicone arm 50 as shown in FIG. 3 for demonstration and testing. In another embodiment, the prototype of FIG. 2 was then added to a Velcro band 120, as shown in FIG. 4, to be applied over a patient's wound.

In an embodiment, when the device 100 was at the proper level of securement there was an audible click, a tactile sensation, and visual feedback. In certain embodiments, the device 100 may include only one of an audible click, a tactile sensation, or visual feedback, or a combination thereof. In an embodiment, the proper level of securement is when the appropriate amount of compression is applied to the patient's wound. In an embodiment, the protocol to use the device of FIGS. 3 and 4 is to tighten the band 115, 120 around a patient's wound area until there is an audible click, secure the band in place, for example securing the Velcro 120 of FIG. 4 in place, and then checking for bleeding, hematomas, or hemostasis patency.

Generally, in an embodiment, for radial hemostasis, a compression over the artery of 100 mmHg (approximately 2 psi compression) is desirable. This level of compression typically creates patent hemostasis and is the amount of pressure necessary to stop bleeding (hemostasis) without completely closing the artery to blood flow. This prevents the condition known as radial artery occlusion. Additionally, patent hemostasis is defined as the proper amount of pressure needed to prevent bleeding and hematoma formation but still allowing some blood flow through the artery to prevent radial artery occlusion.

In the iteration of the prototype device 100 that was constructed, three variables can be manipulated to provide the proper compression: 1) the force needed to cause the audible device to “CLICK”; 2) the area of the patient contact portion 110; and 3) the area of the wrap contacting top 102. In an embodiment, the proper compression force can be applied by manipulating the aforementioned variables. In an embodiment, the force required for hemostasis may vary for different areas of the body, e.g., femoral versus radial sites, venous versus arterial bleeding, or for different patients.

In some embodiments, it will also be possible to distribute different devices, or different parts for the same device to allow the clinician to adjust the device to provide feedback at different levels of compression. This may include but is not limited to varying surface areas, changing lever contact points, or changing the clicker mechanism.

There are many ways to create the audible/tactile feedback. Some of the nonlimiting methods include using a metal that clicks when bent or depressed, a striker and bell-like device if the striker is designed to contact the bell portion after a sufficient force is applied, a cable or zip tie-like mechanism, or an electronic mechanism.

In an embodiment, the audible/tactile feedback device may be used in conjunction with currently marketed inflatable hemostasis devices.

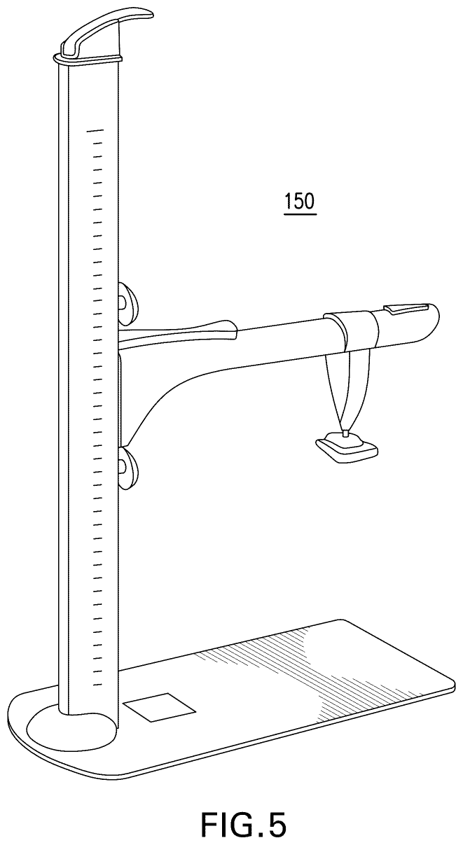

In an embodiment, the audible/tactile feedback device may be used in conjunction with a manual compression or a mechanical device like the CompressAR® device clamp shown in FIG. 5.

In an embodiment, the device will also function over any hemostasis pad or device, like a StatSeal Disc®.

### Testing Examples

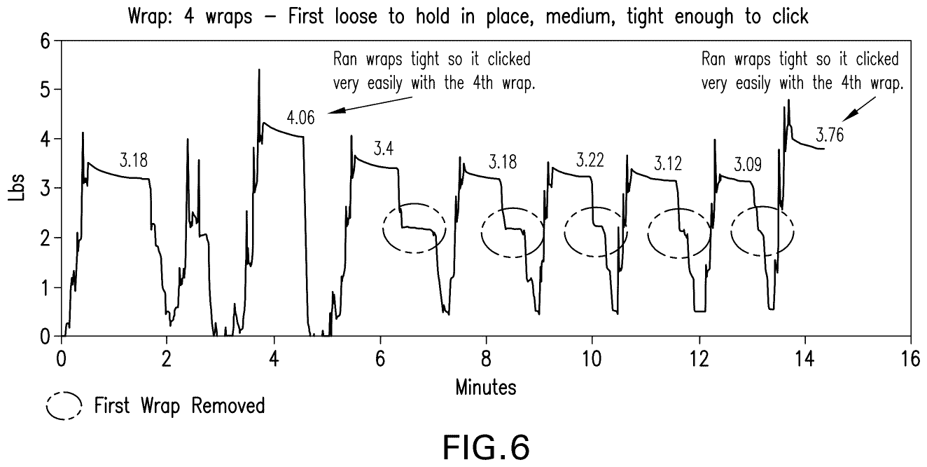

Using the silicone arm model with a load cell inserted at the radial artery position as shown in FIGS. 1-3, compression measure testing was performed. In peaks 1, 4, 5, 6, 7, 8, three medium tightness wraps were applied and then a fourth wrap was applied until the device “clicked”. In peaks 2 and 9, the first three wraps were applied more tightly so that the fourth wrap created the “click” with very light pressure. The results are shown in FIG. 6.

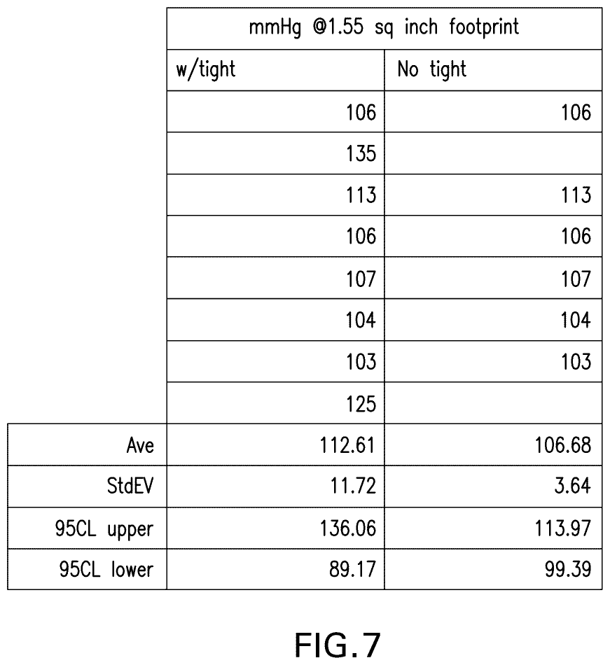

The compression created with the prototype device 100 produced surprisingly consistent results as shown in FIG. 7. As shown, a footprint of 1.55 sq inches is used and the 3 medium wraps followed by the click wrap is analyzed, then the average is 107 mmHg compression with a standard deviation of 3.6 mmHg. This compression force is significantly more consistent than any band that has been tested, due to the variation in how tight bands are applied around a patient's wrist, or variations in inflation levels.

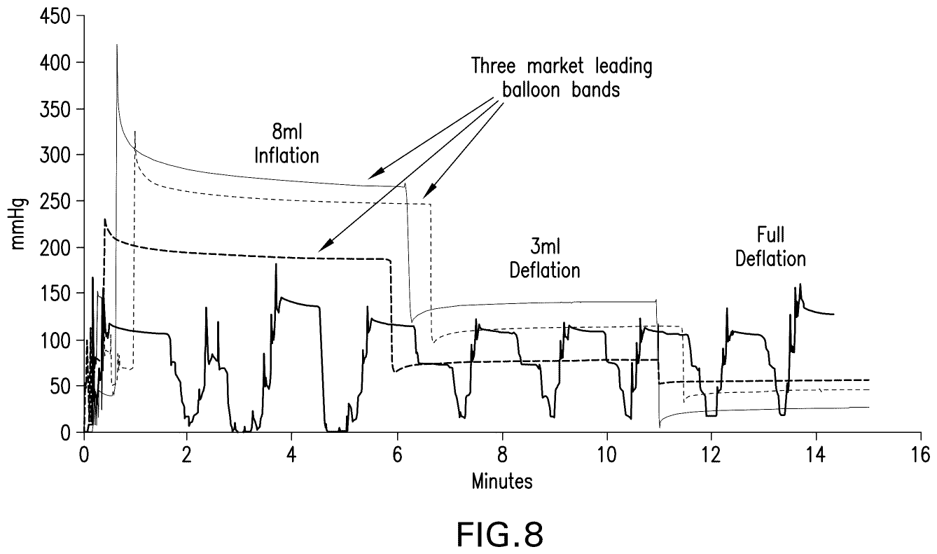

The graph shown in FIG. 8 compares the compression created by the protype of FIG. 3 with three market leading bands using a 2-step deflation protocol. As shown, the compression from the prototype device 100 of the present invention (black graph) is more consistent than the compression created by application and use of the comparable commercial bands at 8 ml inflation, when 3 ml is removed (3 ml deflation), or when the balloon bands are fully deflated.

Generally, known balloon bands on the market typically require multiple interventions by a clinician. In an embodiment, the present device will allow a 1-step protocol for hemostasis that will reduce radial artery occlusions, resulting in a safer use by patients, faster and more consistent hemostasis, and a reduction in the workload for clinicians. It will also save cost in the healthcare setting.

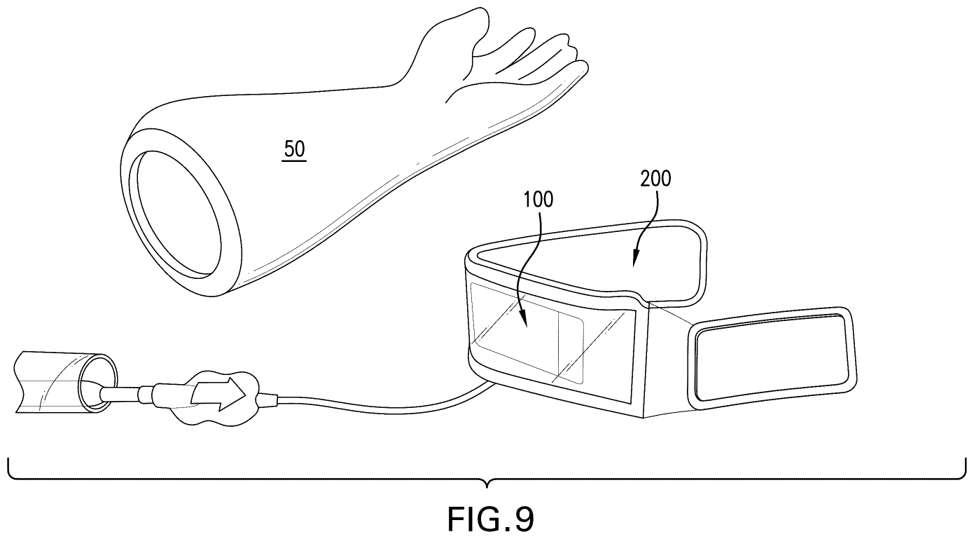

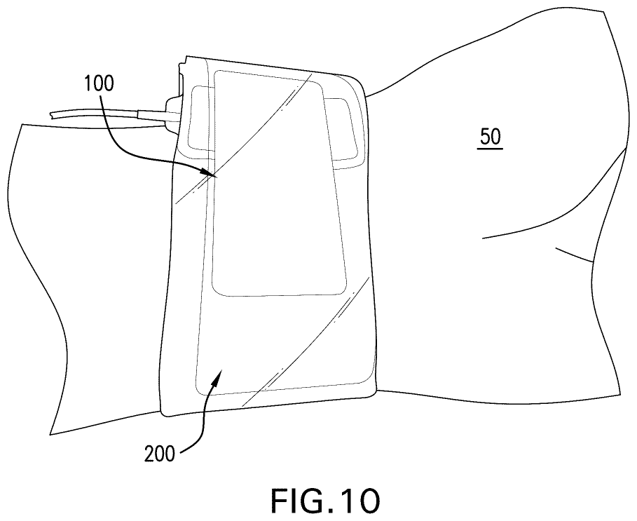

In an embodiment, the mechanism of the device 100 described above can be included with a market leader balloon band 200 (FIG. 9). An example of the method of use for stopping radial bleeding would be to place the balloon band 200 around the patient's wrist. Next, the clinician would inflate the balloon until such time as the balloon presses against the patient's wound and the clicker mechanism, as shown in FIG. 10, until the clicker creates and audible “click.” The compression force where the device clicks is the proper compression for patent hemostasis. In some embodiments, another option would be to design the device so that there is a second step where upon deflation or reduced compression the device “clicks” again creating a lower compression force over the wound.

This device may function with or may be built into any hemostasis device on the market today.

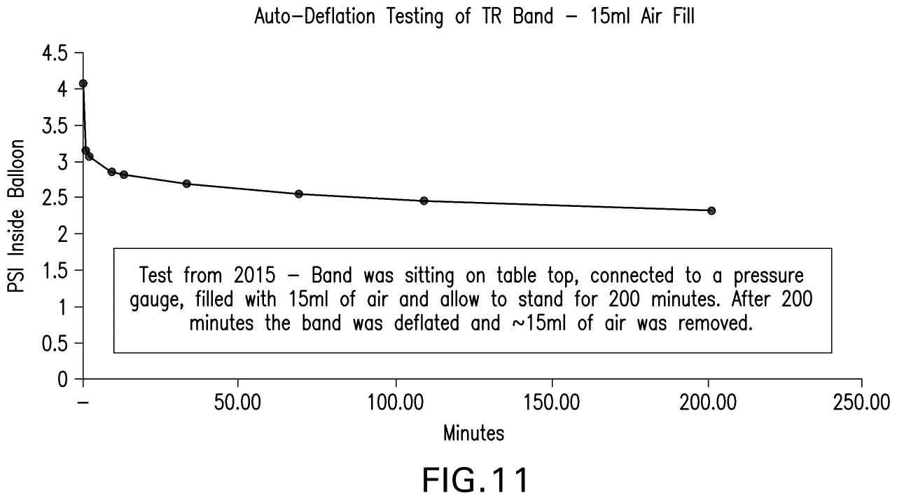

Furthermore, the graph shown in FIG. 11 demonstrates that when the balloon band 200 is filled with air pressure and tested over time, the air pressure within the band is not lost. In an embodiment, the device was inflated to about 15 ml of air and an air pressure gauge was connected to the band. The band was then set on a table top for 200 minutes. At the 200 minutes, all air was removed from the band and the amount of air loss over the 200 minutes period was then measured. As shown, after the 200 minutes, the bad was deflated and about 15 ml of air was removed, thus showing that the air pressure within the band over time was not lost. Additionally, other testing has been conducted in which the band was filled with 30 ml of air and submerged in water and analyzed for air bubbles. In the specific testing, no leakage was seen.

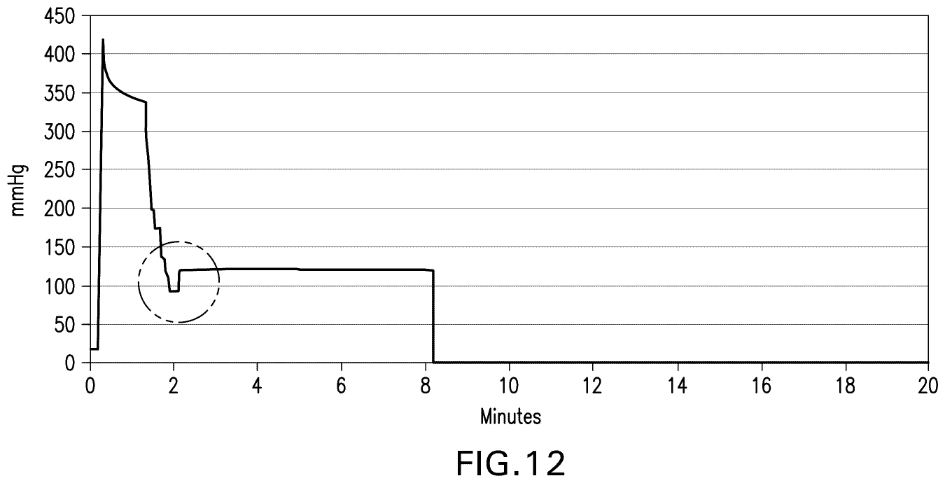

Finally, as shown in FIG. 12, it has been found that deflating about 1 ml of air and adding back in the same amount of the air eliminates some of the band relaxation irregularities. Thus, instead of needing to go back and reassessing for hemostasis patency, a clinician may deflate about 1 ml below patency levels, as shown in the graph as circled, and then reinflate back to the level of patency. This method has shown to stabilize any relaxation irregularities that the band may show over time.

What is claimed is:

1. A device for use with a circumferential wrap, band, or other methods of applying compression over a bleeding wound wherein the device is rigid or semi-rigid and has a patient contact area of 0.5 to 10 square inches comprising a top portion with a wrap contact surface or attachment mechanism to allow the creation of audible and/or tactile feedback at an applied compression force of 50 to 300 mmHg at the patient contact area.

2. The device of claim 1, wherein the device is mechanical.

3. The device of claim 1, wherein the top portion or patient contact area may be changed to vary the compression at the patient contact area.

4. The device of claim 1, wherein the device comprises a lever that may be changed to vary the compression at the patient contact area.

5. The device of claim 1, wherein the compression force is applied manually with a clamp or another mechanical device for creation of the audible and/or tactile response.

6. The device of claim 1, wherein multiple audible and/or tactile feedbacks may be created by applying varying compression forces at varying compressions at the patient contact area.

7. The device of claim 1, wherein the device emits an electronically-created audible feedback when a compression force is applied.

8. The device of claim 1, wherein the device emits multiple electronic audible feedbacks when a compression force is applied.

9. The device of claim 1, wherein the feedback is visual.

10. The device of claim 9, wherein the visual feedback is an illumination of an LED.

11. The device of claim 1, wherein a balloon is used to create the compression causing the audible and/or tactile feedback to signal a compression force is applied.

12. A method of using the device of claim 1 to create hemostasis in a patient.

13. The method of claim 12, wherein the device is used to provide compression feedback when the device is used over a bleeding wound covered with a hemostasis pad.

14. The method of claim 13, wherein hemostasis pad is integral with the device.
